(19) United States
(12) Patent Application Publication
(10) Pub. No.: US 2003/0066535 A1
(43) Pub. Date: Apr. 10, 2003

(54) Multi-point soft restraint apparatus and method

(76) Inventor: Bruce Chapman, Gardiner, NY (US)

Correspondence Address: Edward Etkin, Esq., Suite 3C, 4804 Bedford Avenue, Brooklyn, NY, 11235, US

(21) Appl. No.: 10/265,832

(22) Filed: Oct. 7, 2002

Related U.S. Application Data
(60) Provisional application No. 60/327,671, filed on Oct. 6, 2001.

(51) Int. Cl. A61B 19/00; A61F 5/37

(52) U.S. Cl. 128/869; 128/876

(57) ABSTRACT

An inventive restraint connection system and method of use thereof are provided. The inventive restraint connection system consists of a number of point connector elements that releasably attach to wrist and ankle cuff elements of an existing mechanical restraint system already applied to a subject, that also releasably attach to a stationary or mobile structure, such as a bed or wheelchair. Four point connector elements are typically utilized (two for the wrists and two for the ankles) with an optional fifth leg connector element that releasably centrally attaches to an interlink between ankle cuffs. The point connector elements may be utilized in conjunction with any mechanical restraint system where at least the wrist and ankle cuff modules are supplied with releasable connectors that correspond to the releasable connectors utilized in the point restraint connector elements. During use of the inventive restraint connection system, the restrained subject is placed onto the structure, and the cuff modules (and optionally the ankle interconnect) are releasably connected to the point connector elements by simply snapping in the cuff module connectors into the corresponding connectors on the point connector elements. In an emergency the releasable connectors may be quickly disengaged enabling the subject to be easily removed from the stationary restraint position while advantageously maintaining the application of the existing restraint system to the subject.

### REFERENCE TO PREVIOUSLY FILED APPLICATIONS

The present patent application claims priority from a previously filed commonly assigned U.S. Provisional Patent Application Serial No.: 60/327,671, entitled “Multi-Point Soft Restraint Apparatus and Method” filed on Oct. 6, 2001.

### BACKGROUND OF THE INVENTION

The present invention is directed to a multi-point connector system that, in conjunction with a mechanical restraint system, may be utilized to easily and quickly restrain a subject in an ordinary bed or any other stationary or mobile structure.

There are many thousands of human service and law enforcement agencies and facilities that provide care and supervision to aggressive, suicidal, and emotionally disturbed persons (hereinafter commonly referred to as “EDPs”). The staff and officers working in these agencies regularly come into physical contact with the EDPs through the use of physical subduing or restraint holds when the EDP becomes aggressive. Although there are many types of well-known physical subduing holds, the safest and most advantageous physical subduing hold is a Primary Restraint Technique (PRT) described in greater detail in a commonly assigned co-pending U.S. Patent Application entitled “APPARATUS AND METHOD FOR SAFELY MAINTAINING A RESTRAINING HOLD ON A PERSON”.

While restraint holds are useful for relatively short periods of time, often restraint of the EDP is necessary for an extended period of time. In such cases, the EDP must be restrained using some sort of a mechanical restraint system. Typically, this involves placing wrist and ankle restraints on the EDP so that the EDP may be restrained for an extended period of time at the place of the restraint hold, or, transported to another location while wearing the restraints. Also, for extended restraints, special beds with integrated restraints are used in many facilities. Previously known restraint systems involve mechanical locks—for example, the popular Posey TAT restraint utilizes a mechanical spring-loaded lock that require a special key to open. It takes at least 4-5 staff members to successfully apply such restraints at a speed of no lower than 2-3 minutes per restraint. During application of the restraints, the EDP must be held down and poses a constant threat to the staff members until the restraining process is complete.

Accordingly, removal of such restraints prior to re-application of stationary bed restraints in emergency situations (i.e., in a medical emergency) takes a significant amount of time since a key must be located and used to open each restraint on each limb—this is especially problematic because without the key, which may not be immediately available in case of an emergency, the restraints cannot be removed at all. Manipulation of stationary restraints once attached to the EDP is difficult as well, requiring several people to pull webbing through a complex system of buckles and connectors. Another problem that often arises, is that a bed with integrated mechanical restrains for restraining the EDP for extended periods of time may not be available when necessary, thus forcing the staff members to find alternate long-term restraint means. Finally, complex previously known stationary restraint systems are expensive, heavy, and require extensive training to use properly. This is especially true of beds with integrated restraint systems.

Thus, it would be desirable to provide an apparatus and method for quickly and easily connecting to existing mechanical restraints, already applied to a person, to secure that person to a stationary or mobile object, such as a bed or a wheelchair. It would furthermore be desirable to provide a mechanical restraint connection apparatus that is comfortable to the subject and that may be quickly and easily removed in case of an emergency. It would also be desirable to provide a lightweight mechanical restraint connection system that is easy to transport and use and that is inexpensive to manufacture. Moreover, it would be desirable to provide a mechanical restraint connection system that may be easily controlled by a single person after its application. Finally, it would be desirable to provide a mechanical restraint connection system that may be easily utilized for deployment on beds and other stationary or mobile structures for long term EDP restraint.

### SUMMARY OF THE INVENTION

The apparatus of the present invention, and method of use thereof, remedies the problems associated with securing an already restrained violent and/or struggling EDP to a stationary or mobile structure for long term restraint, and with quickly and easily removing the restrained EDP from the secured position in case of an emergency. In brief summary, the inventive multi-point restraint connection apparatus advantageously provides: (1) quick and easy deployment and connection to an existing mechanical restraint system that has been previously applied to the EDP as well as quick and easy disconnection from the restraint system in case of an emergency; (2) severe restriction to the range of motion of the EDPs arms and legs during long term restraint; (3) a secure fit to any EDP size and any stationary or mobile structure configuration as the inventive restraint connection apparatus is dynamically fitted exactly to the EDP and structure size during application thereof; (4) lightweight and simple construction, making the inventive restraint connectors easy to transport and use, and inexpensive to manufacture; and (5) easy control of the restraint connection apparatus by a single person during and after its application to the EDP.

In summary, the inventive system consists of a number of point connector elements that releasably attach to wrist and ankle cuff elements of an existing mechanical restraint system already applied to the EDP and also releasably attach to a stationary or mobile structure such as a bed or a wheelchair. Four point connector elements are typically utilized (two for the wrists and two for the ankles) with an optional fifth leg connector element that releasably centrally attaches to an interlink between ankle cuffs to further prevent movement of the EDP's legs and lower body subsequent to application of the other point restraint connector elements. The point connector elements may be utilized in conjunction with any mechanical restraint system where at least the wrist and ankle cuff modules are s supplied with releasable connectors that correspond to the releasable connectors utilized in the point restraint connector elements (i.e. male connectors on the cuff modules, if the point restraint connector elements include female releasable connectors, and vice versa).

During use of the inventive point connector elements, the restrained EDP is brought to a structure (such as a bed) where the point connector elements have been already positioned in appropriate locations and removably secured to the object, such that when the EDP is placed onto the object, a staff member can easily releasably connect the cuff modules (and optionally the ankle interconnect) to the point connector elements by simply snapping in the cuff module connectors into the corresponding connectors on the point connector elements. In an emergency the releasable connectors may be quickly disengaged enabling the EDP to be easily removed from the stationary restraint position while advantageously maintaining the application of the existing restraint system to the EDP.

Alternately, the point connector elements may be deployed after the restrained EDP has been brought to the structure and applied to the wrist and ankle cuffs (and optionally interconnect) one at a time.

In one embodiment of the present invention, each point connector element is individually stored and utilized. In another embodiment of the invention, two point connector elements may be connected to one another by elastic webbing or by a tensionable webbing to facilitate quick and easy deployment onto a stationary or mobile structure in preparation for arrival of a restrained EDP. This arrangement also enables the wrist and ankle point connector elements to maintain a desirable position on the structure prior to arrival of the restrained EDP is brought to the structure, the various point connector elements are aligned with their counterpart connectors on the respective wrist and ankle cuffs.

Optionally, a protective sleeve may also be provided for releasable connectors on the wrist point connector elements to prevent the EDP from somehow disengaging the wrist point connector elements. The sleeve enables release of the connectors only by using a long thin object such as a pen perpendicular to the connector (i.e. so a special key is not required). The required angle of use of the object and the required pressure force make it impossible for the EDP to disengage the connector even if the EDP somehow got hold of such an object.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### BRIEF DESCRIPTION OF THE DRAWINGS

In the drawings, wherein like reference characters denote corresponding or similar elements throughout the various figures:

FIG. 1 shows a top view of a point connector element of a first embodiment of the multi-point restraint system of the present invention for releasable connection to wrist or ankle cuff restraints;

FIG. 2 shows a top view of a second embodiment of the present invention in which a pair of the inventive point connector elements of FIG. 1, are connected to one another to facilitate their deployment and positioning on a stationary or mobile structure;

FIG. 3 shows a top view of a point connector element of the multi-point restraint system of the present invention of FIGS. 1 or 2, for releasable connection to an ankle cuff interconnect;

FIG. 4 shows top and side views of an optional releasable connector protective sleeve that may be utilized in conjunction with the point connector elements of FIGS. 1 or 2 to prevent the restrained subject from disengaging the releasable connector;

FIG. 5 shows a top-isometric view of the inventive point connector elements of FIGS. 2 and 3 deployed on a bed in a ready position;

FIG. 6 shows a top-isometric view of the inventive point connector elements of FIGS. 2 and 3 connected to multiple elements of a circular soft restraint system applied to a restrained subject; and

FIG. 7 shows a top-perspective simplified view of mechanical wrist and ankle cuff restraints and interconnects that are applied to a restrained subject and that are used in conjunction with the inventive point connector elements of FIGS. 2 and 3.

### DESCRIPTION OF THE PREFERRED EMBODIMENTS

The present invention relates to a multi-point restraint connector system that, in conjunction with a mechanical restraint system already applied to a subject, may be utilized to easily and quickly restrain a subject in an ordinary bed or in any other stationary or mobile structure (e.g. a wheelchair, a gurney). Preferably, the apparatus and method of the present invention are utilized in conjunction with soft circular restraints, such as those disclosed in the commonly assigned co-pending U.S. patent applications entitled, respectively, “Soft Circular Restraint Apparatus and Method” and “Rapid Deployment Soft Restraint Apparatus and Method” which are hereby both incorporated by reference in their entirety. However, the inventive restraint point connector system may be utilized with other mechanical restraint systems, if the other systems are provided with appropriate releasable interface connectors.

It should be understood, that while the present invention refers to Emotionally Disturbed Persons (hereinafter “EDPs”) and Staff Members, the inventive techniques and apparatus may be applied in virtually any situation where a subject is being restrained and application of mechanical restraints is warranted. Thus, the present invention is applicable in law enforcement, hospitals, mental health care facilities, drug and alcohol rehabilitation centers, etc.

Furthermore, while description of the various embodiments of the present invention refers to connecting to a bed, it should be understood that the inventive point connector elements may be advantageously used to connect to any stationary or mobile structure (such as a wheelchair or a gurney) where it is desirable to maintain the EDP in a restrained position for an extended period of time.

Finally, while a number of exemplary releasable connectors are illustrated in the various drawings and described in conjunction therewith, it should be understood that other types of releasable connectors may be used as a matter of design choice without departing from the spirit of the invention.

Before application of any sort of mechanical restraints, it is important that control over the EDP is established by placing the EDP into a restraining hold. The Primary Restraint Technique (hereinafter “PRT”) is an advantageous modular single person restraint that is applied by an EDP care professional (hereinafter “staff member”) to an EDP from behind. The maneuvers involved in implementing the PRT are described in greater detail in connection with the Primary Restraint Technique (PRT) described in greater detail in a commonly assigned co-pending U.S. Patent Application entitled “APPARATUS AND METHOD FOR SAFELY MAINTAINING A RESTRAINING HOLD ON A PERSON” which is hereby incorporated by reference in its entirety. It should be noted, however, that the restraint system of the present invention does not require use of the PRT—it may be advantageously be utilized in any situation where the EDP is physically restrained by one or more staff members or other individuals.

Referring first to FIG. 7, the multi-point restraint system of the present invention is used in conjunction with wrist cuff restraint system 220 and an ankle cuff restraint system 230. The wrist cuff restraint system 220 is shown in a simplified form includes two wrist cuff restraints 208, 210, each having a respective releasable connector 222, 224, and an optional waist interconnect 216 connecting the wrist cuff restraints 208, 210. The ankle cuff restraint system 230 is also shown in a simplified form and includes two ankle cuff restraints 212, 214, each having a respective releasable connector 234, 236, and an optional ankle interconnect 232 connecting the ankle cuff restraints 212, 214 optionally supplied with a perpendicularly mounted releasable connector 218.

The wrist and ankle cuff restraint systems 220, 230 are preferably ones disclosed in the above-incorporated commonly assigned U.S. patent applications “Soft Circular Restraint Apparatus and Method” and “Rapid Deployment Soft Restraint Apparatus and Method”. However, as a matter of design choice, any mechanical restrain system supplied with releasable connectors 222, 224, 234 and 236 (and optionally connector 218) or the like, may be readily utilized in conjunction with the inventive multi-point restraint connection system, without departing from the sprit of the invention.

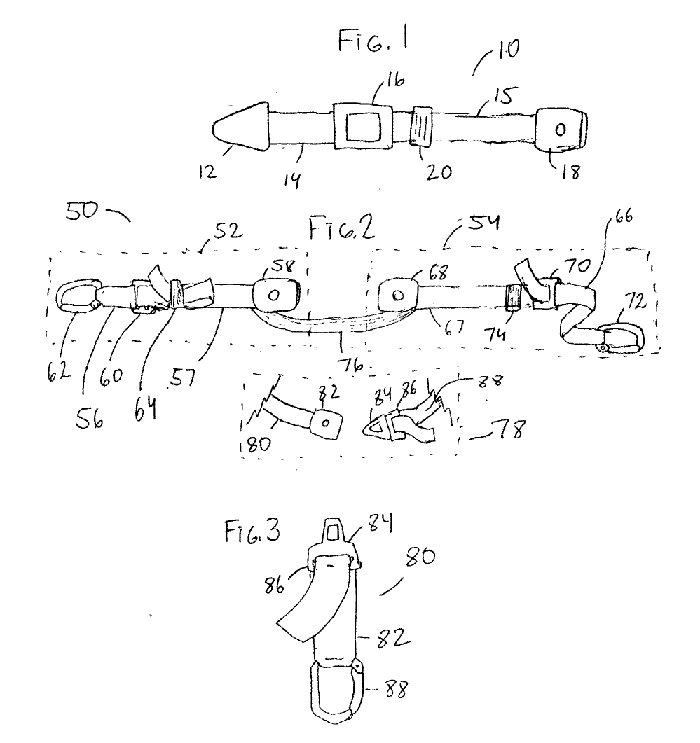

Referring now to FIG. 1, a first embodiment of the inventive multi-point restraint system is shown as a point connector element 10. The point connector element 10 comprises a releasable point connector 12 for releasably connecting to a stationary or mobile structure, such as a bed frame (not shown), at one end, a first webbing element 14 connected to a tensioning mechanism 16 and a second webbing element 15 connecting the tensioning mechanism 16 to a releasable restraint connector 18. An optional elastic loop 20 may be positioned between the tensioning mechanism 16 and the releasable connector 18 for retaining a folded potion of the webbing element 15 that passes through the tensioning mechanism 16 and that is used to adjust the length of the point connector element 10.

The tensioning mechanism 16 can be used to control the overall length of the point connector element 10. The webbing elements 14 and 15 are preferably composed of a strong flexible material such as synthetic webbing (for example, the nylon webbing used in seat belts and parachute straps) or a similar strong flexible material having high tensile strength. The tensioning mechanism 16 may be any buckle-type tensioning device composed of rigid plastic or metal. The releasable point connector 12 may be any openable releasable connector (such as a carabiner-type connector) capable of connecting to a bed frame or the like. Optionally, the point connector 12 and a portion of the webbing element 14 may be looped around a frame or another portion of a structure on which the EDP is being restrained (not shown) and releasably connected back to the webbing element 14 forming a noose-like loop.

The restraint connector 18 is configured for and used to releasably connect to a corresponding releasable connector 222, 224 on the wrist cuff system 220 or to a corresponding releasable connector 234, 236 on the ankle cuff system 230 (see FIG. 7). The releasable connector 18 and the corresponding releasable connectors 222, 224, 234, and 236, may be any matched releasable mechanical connector such as a snap buckle, or a male/female seatbelt-type connector. For example, if the connectors 222, 224, 234, and 236 are male seatbelt-type connectors, the releasable connector 18 is a female seatbelt-type connector.

During use of the inventive multi-point restraint connector system, four point connector elements 10 are preferably utilized—two for wrist cuff restraint system 220 and two for the ankle cuff restraint system 230. The point connectors 12 are releasably connected to the structure on which the EDP is being restrained (not shown) while the restraint connectors 18 are connected to the corresponding releasable connectors 222, 224, 234, and 236 such that the EDP is releasably restrained at the stationary or mobile structure. In case of an emergency, the EDP can be easily and quickly removed from the structure without disengaging the wrist and ankle restraint systems 220, 230 by simply disengaging the restraint connectors 18 on each point connector elements 10.

Referring now to FIG. 2, a second embodiment of the present invention is shown as a dual point restraint connector system 50 that may be utilized either with the wrist restraint system 220, or with the ankle restraint system 230. The connector system 50 includes two point connector elements 52 and 54 connected to one another via an interconnect 76. Each of the point connector elements 52 and 54 are substantially identical to the point connector element 10 of FIG. 1 and include components that correspond to equivalents on the point connector element 10. Thus, releasable point connectors 62, 72 correspond to the releasable point connector 12, webbing elements 56, 66 correspond to the webbing element 14 (FIG. 1), webbing elements 57, 67 correspond to the webbing element 15 (FIG. 1), tensioning mechanisms 60, 70 correspond to the tensioning mechanism 16, releasable restraint connectors 58, 68 correspond to the releasable restraint connector 18; and the elastic loops 64, 74 correspond to the elastic loop 20.

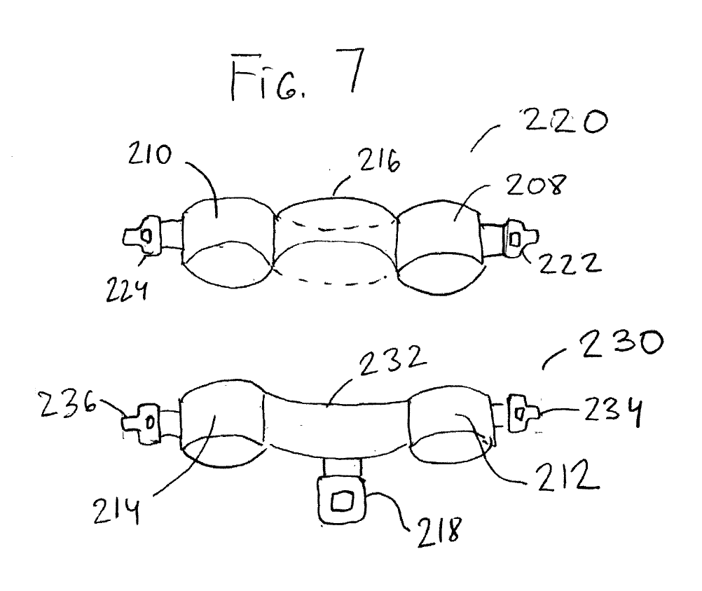

The interconnect 76 is preferably a flexible (and optionally elastic) member secured to undersides of webbing elements 57, 67 proximal to the respective releasable restraint connectors 58, 68. The main purpose of the interconnect 76 is to keep the point connector elements 52 and 54 in pairs and to facilitate ready deployment on a stationary or mobile structure (not shown) prior to arrival of the restrained EDP. This functionality of the interconnect 76 is described in greater detail below in connection with FIG. 5. The interconnect 76 may be composed of any flexible resilient material such as rubber or rubberized material, stretch webbing, or a non-stretch synthetic webbing. Optionally, if the interconnect 76 is not elastic, it may be supplied with a tensioning mechanism (not shown) to control its length.

An alternate embodiment of the interconnect 76 is shown in FIG. 2 as releasable interconnect 78. The releasable interconnect 78 includes a first webbing element 80 secured by its first end to one of the webbing elements 57, 67 and a is second webbing element 88 secured by its first end to the other of the webbing elements 57, 67. The second ends of the elements 80, 88 include matched releasable connectors 82, 84 configured for releasable connection to one another. The releasable connectors 82, 84 may be snap buckles or a male/female seatbelt-type connector system. A tensioning mechanism 86 is provided on or both one of 20 the elements 80, 88 to control the length of the releasable interconnect 78 (only one tensioning mechanism is shown in FIG. 2). The releasable interconnect 78 may be used to disconnect the point connector elements 52 and 54 in case one of them becomes damaged or needs to be replaced.

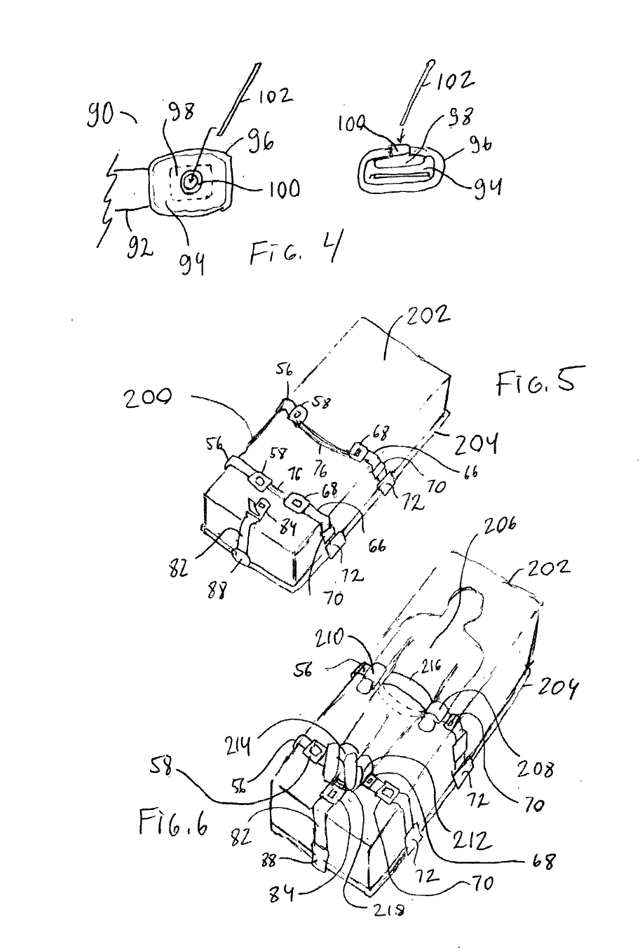

Referring now to FIG. 3, an optional leg tensioning point element 80, that may be used in conjunction with the inventive embodiments of FIGS. 1 and 2 is shown. The tensioning point element 80 includes a releasable point connector 88, such as a carabineer, at one end, a webbing element 82 connecting the releasable point connector 88 to a tensioning mechanism 86 with an integrated releasable restraint connector 84 at the other end configured to releasably connect to a corresponding releasable connector 218 disposed perpendicular to the interconnect 232 on the ankle restraint system 230. Optionally, the tensioning mechanism 86 may be a separate device from the releasable restraint connection 84. The tensioning point element 80 may be advantageously utilized to further restrict movement of the EDPs legs and lower body once the EDP has been secured to a stationary or mobile structure (not shown) by connecting the releasable connectors 84 and 218, connecting the releasable point connector 88 to a stationary or mobile structure (not shown), and then adjusting the tensioning mechanism 86 to secure the EDPs legs to the structure as tightly as possible and to prevent lateral and vertical movement thereof.

Referring now to FIG. 4, an optional secure releasable connector 90 is shown. The secure releasable connector 90 may be used when the point connector elements 10, 52, 54 utilize seatbelt-type female connectors as a replacement for the releasable connectors 18, 58, 58. While seatbelt-type releasable connectors are easy to use and easy to release in case of an emergency, they may become accessible to a particularly flexible or clever EDP and thus pose a risk of EDP disengaging themselves from the connectors by pressing release buttons (not shown) with their fingers. The secure releasable connector 90 solves this challenge by proving a protective sleeve over a standard seatbelt-type female connector and restricting access to the release button.

The secure releasable connector 90 is connected to a webbing element 92 (corresponding to one of the webbing elements 15, 57 or 67) and includes a female seatbelt-type connector 94 with a release button 98. The connector 94 is encased in a protective sleeve 96 (for example composed of plastic or a synthetic material such as nylon) with a small opening 100 positioned over the release button 98. The opening 100 is preferably too small for a finger and thus a long thin object 102 (such as a pen or a pencil) is necessary to disengage the connector 94 by passing the object 102 through the opening 100 until the release button 98 is sufficiently depressed. The force necessary for activation of the release button 98 and the angle of entry of the object 102 make it impossible for the EDP to disengage the connector 90, even if the EDP somehow obtains the object 102. Because the EDPs can only potentially access point connector elements that proximal to their hands, the secure connector 90 is only useful in point connector elements 10 or 52, 54 that are used in conjunction with the wrist restraint system 220.

Referring now to FIG. 5, the inventive multi-point restraint connector system of FIGS. 2 and 3 is shown as deployed (and ready to receive the restrained EDP) on a bed 200 having a mattress 202 and a circumferential frame 204. Two sets of point restraint systems 50 are positioned on the mattress 202, one for each wrist and ankle restraint system 220, 230, and a tensioning point element 80 is positioned at the foot of the bed 200 ready to releasably connect to the ankle restraint system 230. The interconnects 76 advantageously enable the point restraint systems 50 to be easily positioned in desirable locations on the mattress 202 without the possibility of the individual point connector elements 52, 54 sliding off the mattress 202 either prior to or after the EDP is brought to the bed 200.

Typically, one staff member prepares the point restraint systems 50 and the tensioning point element 80 by connecting the respective connectors 62, 72 and 88 to the bed frame 204, while one or more other staff members escort the EDP, already in wrist and ankle restraints 220, 230, to the bed 200 and place the EDP thereon.

Referring now to FIG. 6, the EDP 206 is positioned on the bed 200 and the various releasable connector elements 58, 68, and 84 are connected to corresponding releasable connectors of the restraint systems 220, 230 disposed on the EDP 206. The EDP 206 is thereby restrained on the bed 200 and may be quickly removed from the bed 200 for transport or in case of an emergency without releasing the EDP 206 from the restraint systems 220, 230. This is particularly advantageous because in all previously known systems, the mechanical restraints were incorporated into the bed and, if the EDP had to be moved, the staff members were forced to release the EDP from the bed restraints before applying separate transport restraints, thus putting themselves and the EDP at risk. Furthermore, in case of am emergency, not only are previously known bed restraints slow to remove, but also the EDP will be left unrestrained if there is no time to apply a new set of restraints (such as in case of a fire).

It should be noted that the specific structures and appearance of the elements and components of FIGS. 1-7 are shown by way example only and shall not serve as a limitation on structure and functionality of the inventive elements.

All together, the lightweight inventive point connector restraint elements 10, 52, 54, 80 can withstand approximately 10,000 pounds of force, all without any metal key elements. Because all elements of the inventive systems are flexible and soft, the EDP will not hurt themselves or experience significant discomfort by struggling against the restraints. It takes less than 25 seconds for a single person to deploy each of the inventive arm and leg restraint systems from a storage position to their deployed restraint position. Most importantly, the inventive point connector elements 10, 52, 54, 80 can be easily released in several second in an emergency situation without need for a special key. Moreover, the multi-point restraint connection system of the present invention is easy and intuitive to use and inexpensive to manufacture. Finally, the construction of the inventive multi-point restraint connection system makes it easy to deploy in any setting and with any stationary or mobile structure, such as a bed, a wheelchair, or a gurney even one that is not designed for use with restraints.

Thus, while there have been shown and described and pointed out fundamental novel features of the invention as applied to preferred embodiments thereof, it will be understood that various omissions and substitutions and changes in the form and details of the devices and methods illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention.

I claim:

1. An apparatus for releasably connecting a person restrained with a mechanical restraint system to a structure, the mechanical restraint system, comprising a plurality of limb cuff restraints each having a releasable cuff connector, the apparatus comprising:
a plurality of point connector elements operable to releasably connect to corresponding plurality of limb cuff restraints each having a releasable cuff connector, each of said plural point restraint connector elements comprising:
a first releasable point connector operable to releasably connect to the structure;
a releasable restraint connector operable to releasably connect to one of the plural releasable cuff connectors;
a first tensioning device operable to adjust a length of said plural point connector element;
a first elongated webbing element connecting said releasable point connector to said first tensioning device; and
a second elongated webbing element connecting said first tensioning device to said restraint connector, wherein when the restrained person is positioned on the structure, each said plural point restraint connector element is releasably connected to the structure through said point releasable connector and also releasably connected to one of the corresponding plural releasable cuff connector, thus releasably connecting the restrained person to the structure.

2. The apparatus of claim 1, further comprising an elongated interconnect element connecting each pair of said plural point restraint connector elements to one another, said interconnect element comprising a first end connected to an underside of said second webbing element and proximal to said releasable restraint connector of one of said pair of plural point restraint connector elements and a second end connected to an underside of said second webbing element and proximal to said releasable restraint connector of said other of said pair of plural point restraint connector elements.

3. The apparatus of claim 2, wherein said interconnect element is composed of an elastic material.

4. The apparatus of claim 2, wherein said interconnect element comprises at least one of a tensioning mechanism operable to control an length thereof, and a releasable interconnect coupling operable to disconnect said plural point restraint connector elements from one another.

5. The apparatus of claim 1, wherein the mechanical restraint system comprises an ankle interconnect element having a first releasable connector mounted thereon, further comprising a leg point restraint connector element comprising:
a second releasable connector sized and configured to releasably attach to said first releasable connector;
a second releasable point connector operable to releasably connect to the structure;
a second tensioning device operable to adjust a length of said leg point connector element; and
a third elongated webbing element connecting said second releasable connector to said second tensioning device, wherein when the restrained person is positioned on the structure, said leg point restraint connector element is releasably connected to the structure through said point releasable connector and also releasably connected to the first releasable connector to restrict lateral and vertical motion of the restrained person's legs.

6. The apparatus of claim 1, further comprising an elastic loop positioned on said second webbing element between said first tensioning device and said releasable restraint connector, operable to retain a folded potion of said second webbing element that passed through said first tensioning device.

7. The apparatus of claim 1, wherein the releasable cuff connector comprises one of a male and female seatbelt-type connector, and wherein said releasable restraint connector comprises the other of said a male and female seatbelt-type connector.

8. The apparatus of claim 1, wherein said first and second webbing elements are composed of high tensile strength synthetic webbing.

9. The apparatus of claim 5, wherein said third webbing element is composed of high tensile strength synthetic webbing.

10. The apparatus of claim 7, wherein said female seatbelt-type connector comprises a release button operable to disengage said female seatbelt-type connector from said corresponding male seatbelt-type connector, and a protective sleeve that restricts access to said release button through a hole defined in said sleeve over said release button, sized and configured to restrict passage of elongated objects therethrough that are smaller than a human finger, wherein female seatbelt-type connector may only be disengaged by passing an elongated object through said hole in a direction of said release button until said release button is activated.

11. The apparatus of claim 1, wherein said first releasable point connector comprises a carabiner-type connector.

12. A method for releasably connecting a person restrained with a mechanical restraint system to a structure, the mechanical restraint system, comprising a plurality of limb cuff restraints each having a releasable cuff connector, comprising the steps of:
(a) providing a plurality of point connector elements operable to releasably connect to corresponding plurality of limb cuff restraints each having a releasable cuff connector, each of said plural point restraint connector elements comprising a first releasable point connector operable to releasably connect to the structure; a releasable restraint connector operable to releasably connect to one of the plural releasable cuff connectors; a first tensioning device operable to adjust a length of said plural point connector element; a first elongated webbing element connecting said releasable point connector to said first tensioning device; and a second elongated webbing element connecting said first tensioning device to said restraint connector,
(b) releasably connecting each said plural point restraint connector element to the structure through each said point releasable connector;
(c) positioning the restrained person on the structure to align said plural releasable cuff connectors with corresponding releasable restraint connectors; and
(d) releasably connecting each said plural releasable cuff connector with a corresponding releasable restraint connector, thus releasably securing the restrained person to the structure.
